SEQUENCE LISTING (1 sequence)

SEQ ID NO: 1 - DNA, 6293 bp, Artificial Sequence
  Temperature sensitive construct cm5
catgatgacaataaagaattaattattgttcactttattcgactttaatatatccatcacgttagaaaatgcgatatcgcgacgaggatctatgtatctaataggatctattgcggtggtagctagagaggattcttttttgaatcgcatcaaactaatcacaaagtcgaacaaatatcctttattaagtttgacccttccatctgtaacaatagggaccttgttaaacagttttttaaaatcttgagagtctgtgaattttgtcaattgtctgtattcctctgaaagagattcataacaatgacccacggcttctaatttattttttgattggatcaataataataacagaaagtctagatattgagtgatttgcaatatatcagataatgaagattcatcatcttgactagccaaatacttaaaaaatgaatcatcatctgcgaagaacatcgttaagagatactggttgtgatccatttattgatcgcaaaagcttaaaaattgaaattttattttttttttttggaatataaatacatattgaagagacagagtgatatataaaactgctaaaatgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgaagaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgtcaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgacgtacgtcactttttataaaatatagtagaatttcattttgtttttttctatgctataaataggaattgtgagcgctcacaattccaaaatggttcactgtgctggatgcgaaaggcccattctggaccgtttcttgttgaatgtgctggacagggcttggcatgtgaaatgcgtgcagtgctgtgagtgtaagtgcaacctgaccgagaaatgcttctccagggagggcaagctctactgtaaaaacgactttaaagaaatagaattaattggctcaggtggatttggccaagttttcaaagcaaaacacagaattgacggaaagacttacgttattaaacgtgttaaatataataacgagaaggcggagcgtgaagtaaaagcattggcaaaacttgatcatgtaaatattgttcactacaatggctgttgggatggatttgattatgatcctgagaccagtgatgattctcttgagagcagtgattatgatcctgagaacagcaaaaatagttcaaggtcaaagactaagtgccttttcatccaaatggaattctgtgataaagggaccttggaacaatggattgaaaaaagaagaggcgagaaactagacaaagttttggctttggaactctttgaacaaataacaaaaggggtggattatatacattcaaaaaaattaattcatagagatcttaagccaagtaatatattcttagtagatacaaaacaagtaaagattggagactttggacttgtaacatctctgaaaaatgatggaaagcgaacaaggagtaagggaactttgcgatacatgagcccagaacagatttcttcgcaagactatggaaaggaagtggacctctacgctttggggctaattcttgctgaacttcttcatgtatgtgacactgcttttgaaacatcaaagtttttcacagacctacgggatggcatcatctcagatatatttgataaaaaagaaaaaactcttctacagaaattactctcaaagaaacctgaggatcgacctaacacatctgaaatactaaggaccttgactgtgtggaagaaaagcccagagaaaaatgaacgacacacatgttagtccaaacccacccgctttttatagtaagtttttcacccataaataaaaatgtcgtttactttgaccaacaagaacgtgattttcgttgccggtctgggaggcattggtctggacaccagcaaggagctgctcaagcgcgatcccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtaacctatcccattacggtcaatccgccgtttgttcccacggagaatccgacgggttgttactcgctcacatttaatgttgatgaaagctggctacaggaaggccagacgcgaattatttttgatggcgttaactcggcgtttcatctgtggtgcaacgggcgctgggtcggttacggccaggacagtcgtttgccgtctgaatttgacctgagcgcatttttacgcgccggagaaaaccgcctcgcggtgatggtgctgcgttggagtgacggcagttatctggaagatcaggatatgtggcggatgagcggcattttccgtgacgtctcgttgctgcataaaccgactacacaaatcagcgatttccatgttgccactcgctttaatgatgatttcagccgcgctgtactggaggctgaagttcagatgtgcggcgagttgcgtgactacctacgggtaacagtttctttatggcagggtgaaacgcaggtcgccagcggcaccgcgcctttcggcggtgaaattatcgatgagcgtggtggttatgccgatcgcgtcacactacgtctgaacgtcgaaaacccgaaactgtggagcgccgaaatcccgaatctctatcgtgcggtggttgaactgcacaccgccgacggcacgctgattgaagcagaagcctgcgatgtcggtttccgcgaggtgcggattgaaaatggtctgctgctgctgaacggcaagccgttgctgattcgaggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctaccggcgatgagcgaacgcgtaacgcgaatggtgcagcgcgatcgtaatcacccgagtgtgatcatctggtcgctggggaatgaatcaggccacggcgctaatcacgacgcgctgtatcgctggatcaaatctgtcgatccttcccgcccggtgcagtatgaaggcggcggagccgacaccacggccaccgatattatttgcccgatgtacgcgcgcgtggatgaagaccagcccttcccggctgtgccgaaatggtccatcaaaaaatggctttcgctacctggagagacgcgcccgctgatcctttgcgaatacgcccacgcgatgggtaacagtcttggcggtttcgctaaatactggcaggcgtttcgtcagtatccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccagcgctgacggaagcaaaacaccagcagcagtttttccagttccgtttatccgggcaaaccatcgaagtgaccagcgaatacctgttccgtcatagcgataacgagctcctgcactggatggtggcgctggatggtaagccgctggcaagcggtgaagtgcctctggatgtcgctccacaaggtaaacagttgattgaactgcctgaactaccgcagccggagagcgccgggcaactctggctcacagtacgcgtagtgcaaccgaacgcgaccgcatggtcagaagccgggcacatcagcgcctggcagcagtggcgtctggcggaaaacctcagtgtgacgctccccgccgcgtcccacgccatcccgcatctgaccaccagcgaaatggatttttgcatcgagctgggtaataagcgttggcaatttaaccgccagtcaggctttctttcacagatgtggattggcgataaaaaacaactgctgacgccgctgcgcgatcagttcacccgtgcaccgctggataacgacattggcgtaagtgaagcgacccgcattgaccctaacgcctgggtcgaacgctggaaggcggcgggccattaccaggccgaagcagcgttgttgcagtgcacggcagatacacttgctgatgcggtgctgattacgaccgctcacgcgtggcagcatcaggggaaaaccttatttatcagccggaaaacctaccggattgatggtagtggtcaaatggcgattaccgttgatgttgaagtggcgagcgatacaccgcatccggcgcggattggcctgaactgccagctggcgcaggtagcagagcgggtaaactggctcggattagggccgcaagaaaactatcccgaccgccttactgccgcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagtcaacagcaactgatggaaaccagccatcgccatctgctgcacgcggaagaaggcacatggctgaatatcgacggtttccatatggggattggtggcgacgactcctggagcccgtcagtatcggcggaattacagctgagcgccggtcgctaccattaccagttggtctggtgtcaaaaataaaatcattcctaaattatagaaatgatctctcaaataacgtatatgtgtaccaggagccgatcctatatacactacggtggcaccatctaatataccgtgtcgctgtaacttactaagaaaaaataattctcctagtaatagttttaactgtccttgatacggcagtttttttgcgacctcatttgcactttctggttcgtaatctaactcattatcaatttcctcaaaatacataaacggtttatctaacgacacaacatccatttttaagtattatattaaaatttaatcaatgtttatttttagttttttagataaaaaatataatatta

(19) United States
(12) Patent Application Publication
(10) Pub. No.: US 2015/0240246 A1
(43) Pub. Date: Aug. 27, 2015

(54) Temperature-dependent insertion of genetic material into genomic DNA

(71) Applicants: Bertram JACOBS, Tempe, AZ (US); Karen KIBLER, Tempe, AZ (US)

(72) Inventors: Bertram JACOBS, Tempe, AZ (US); Karen KIBLER, Tempe, AZ (US)

(21) Appl. No.: 14/707,252

(22) Filed: May 8, 2015

Related U.S. Application Data
(62) Division of application No. 14/149,398, filed on Jan. 7, 2014.
(60) Provisional application No. 61/750,194, filed on Jan. 8, 2013.

(51) Int. Cl. C12N 15/66

(52) U.S. Cl. CPC C12N 15/66

(57) ABSTRACT

The present invention provides improved methods and reagents for insertion of genetic material into genomic DNA.

### CROSS-REFERENCE

This application claims is a divisional of U.S. application Ser. No. 14/149,398 filed Jan. 7, 2014, which claims priority to U.S. Provisional Patent Application Ser. No. 61/750,194 filed Jan. 8, 2013, incorporated by reference herein in its entirety.

### BACKGROUND

Though progress has been made in making vaccines available to prevent human disease, we still lack preventive measures for many of the human pathogens. For decades, we have seen pox viruses go through many revisions as vaccine vectors: they have been attenuated, made replication-competent, and modified to alter immune response. Each time a new vector is prepared, technology must be used to insert the antigen of interest. Current methods to ensure that only viruses having replaced the original sequence with the target gene will survive are very time consuming and are not deemed compatible with the FDA requirements.

### SUMMARY OF THE INVENTION

In a first aspect, the invention provides methods for insertion of genetic material into genomic DNA comprising

(a) incubating host cells comprising recipient viral vector and donor nucleic acid vector under conditions and for a time suitable for promoting recombination between the recipient viral vector and the donor nucleic acid vector, wherein
(i) the recipient viral vector comprises
   (A) a gene encoding a toxic protein, wherein expression of the toxic protein is temperature sensitive; or the toxic protein itself is only toxic at a particular temperature; and
   (B) two recombination sites flanking the gene encoding the toxic protein, wherein the two recombination sites are unique in the recipient viral vector; wherein
(ii) the donor nucleic acid vector comprises
   (A) a donor gene; and
   (B) two recombination sites flanking the donor gene, wherein the two recombination sites are (I) unique in the donor nucleic acid vector, and (II) identical to the two recombination sites in the recipient viral vector; and wherein
(iii) the host cell comprises a promiscuous DNA polymerase capable of synthesizing recipient viral vector DNA, and

wherein the conditions comprise incubating the host cells at a first temperature at which the toxic protein is not expressed;

(b) subsequently culturing the host cells at a second temperature at which the toxic protein is expressed by the recipient viral vector; and

(c) selecting viruses that grow at the second temperature, wherein viruses that grow at the second temperature are recombinant recipient viral vectors in which the donor gene has been inserted into the recipient viral vector, replacing the toxic gene.

In a second aspect, the invention provides nucleic acid cassettes, comprising:

(a) a gene encoding a toxic protein;

(b) a regulatory element operatively linked to the gene encoding the toxic protein; and

(c) a temperature sensitive repressor of the regulatory element.

In a further aspect, the invention provides recombinant viral vectors, comprising

(a) a gene encoding a toxic protein, wherein expression of the toxic protein is temperature sensitive; and

(b) two recombination sites flanking the gene encoding the toxic protein, wherein the two recombination sites are unique in the recipient viral vector.

In a further embodiment, the recombinant viral vectors further comprise:

(c) a regulatory element operatively linked to the gene encoding the toxic protein; and

(d) a temperature sensitive repressor of the temperature sensitive regulatory system.

In another aspect, the present invention provides recombinant viral particles comprising the recombinant viral vectors of the invention.

### BRIEF DESCRIPTION OF THE FIGURES

FIG. 1 is an exemplary diagram of a transfer plasmid according to the invention.

FIG. 2 is a diagram of an exemplary recombination event according to the invention.

FIG. 3 is a diagram of an exemplary recombination event according to the invention.

### DETAILED DESCRIPTION OF THE INVENTION

As used in the specification, the singular forms “a,” “an” and “the” include plural referents unless the context clearly dictates otherwise.

Any terms not directly defined herein shall be understood to have the meanings commonly associated with them as understood within the art of the present disclosure. Certain terms are discussed herein to provide additional guidance to the practitioner in describing the compositions, devices, methods, and the like, of embodiments of the present disclosure, and how to make or use them. It will be appreciated that the same thing can be said in more than one way. Consequently, alternative language and synonyms can be used for any one or more of the terms discussed herein. No significance is to be placed upon whether or not a term is elaborated or discussed herein. Some synonyms or substitutable methods, materials and the like are provided. Recital of one or a few synonyms or equivalents does not exclude use of other synonyms or equivalents, unless it is explicitly stated. Use of examples, including examples of terms, is for illustrative purposes only and does not limit the scope and meaning of the embodiments of the present disclosure herein.

All embodiments disclosed herein can be used in combination, unless the context clearly indicates otherwise. Unless the context clearly requires otherwise, throughout the description and the claims, the words ‘comprise’, ‘comprising’, and the like are to be construed in an inclusive sense, as opposed to an exclusive or exhaustive sense; that is to say, in the sense of “including, but not limited to”. Words using the singular or plural number also include the plural or singular number, respectively. Additionally, the words “herein,” “above” and “below” and words of similar import, when used in this application, shall refer to this application as a whole and not to any particular portions of this application.

In a first aspect, the invention provides gene insertion methods comprising

(a) incubating host cells comprising recipient viral vector and donor nucleic acid vector under conditions and for a time suitable for promoting recombination between the recipient viral vector and the donor nucleic acid vector, wherein
(i) the recipient viral vector comprises
   (A) a gene encoding a toxic protein, wherein expression of the toxic protein is temperature sensitive; or the toxic protein itself is only toxic at a particular temperature; and
   (B) two recombination sites flanking the gene encoding the toxic protein, wherein the two recombination sites are unique in the recipient viral vector; wherein
(ii) the donor nucleic acid vector comprises
   (A) a donor gene; and
   (B) two recombination sites flanking the donor gene, wherein the two recombination sites are (I) unique in the donor nucleic acid vector, and (II) identical to the two recombination sites in the recipient viral vector; and wherein
(iii) the host cell comprises a promiscuous DNA polymerase capable of synthesizing recipient viral vector DNA, and

wherein the conditions comprise incubating the host cells at a first temperature at which the toxic protein is not expressed;

(b) subsequently culturing the host cells at a second temperature at which the toxic protein is expressed by the recipient viral vector; and

(c) selecting viruses that grow at the second temperature, wherein viruses that grow at the second temperature are recombinant recipient viral vectors in which the donor gene has been inserted into the recipient viral vector, replacing the toxic gene.

The proposed method is a shift in concept: the parental recipient virus itself will be nonviable unless it has undergone recombination to replace the DNA encoding the toxic protein, with the donor gene. There are no substances that are toxic to the cells in the resulting recombinant virus, and there are no substances that increase the rate of mutation in the virus. There are no materials utilized that pose a risk to humans; the entire selection is for replication at the second temperature. Yet the time required to acquire the recombinant virus that expresses the donor gene will be a fraction of that needed for the methods currently used.

Any suitable host cells can be used that are capable of infection by the recipient viral vector to be used, and can have the donor nucleic acid to be used introduced into the cell. The host cell is preferably a eukaryotic cell, and more preferably a mammalian cell, including but are not limited to BSC-40 cells, BHK cells, MRC-5 cells, Vero cells, RK-13 cells, and CEFs.

Any suitable recipient viral vector may be used that can infect the host cells being used, including but not limited to a Pox viral genome, including but not limited to vaccinia viral genome and derivatives of vaccinia viral genomes. The recipient viral vector is a recombinant vector, where a portion of the viral genome has been replaced with a “cassette” including the toxic protein. Any suitable site in the viral genome may be replaced, including but not limited to the thymidine kinase locus in vaccinia virus and derivatives thereof. Pox viruses can survive and replicate without thymidine kinase.

The recipient viral vector comprises a gene encoding a toxic protein whose expression is lethal to the viral vector (or lethal to the host cell that is required for viral replication), wherein expression of the toxic protein is temperature sensitive. Alternatively, the recipient viral vector comprises a gene encoding a toxic protein that is itself temperature sensitive. Any gene encoding a suitable toxic protein can be used, including but not limited to genes encoding protein kinase R (PKR), DNAses (including but not limited to DNAse I, DNAse II, and micrococcal nuclease), RNAses (including but not limited to RNAse-A, RNAse-T1, and RNAse-T2), restriction endonucleases (including but not limited to Type I such as M.EcoK1, Type II such as EcoRI or HindIII or NotI, Type III such as PstII, and synthetic zinc finger nucleases), proteases (including but not limited to Proteinase K), and genes that induce apoptosis (including but not limited to PUMA, or bax, or bac), or modifications of these genes. Such genes are known to those of skill in the art, while those of skill in the art will readily understand how to utilize newly identified genes encoding toxic proteins in the methods of the invention, based on the teachings herein.

In one embodiment, expression of the gene encoding the toxic protein is controlled by a heterologous temperature sensitive regulatory system, wherein the temperature sensitive element is present, or is encoded by a nucleic acid present, between the two recombination sites. In one embodiment, the heterologous temperature sensitive regulatory element comprising or consisting of a temperature sensitive lac repressor, wherein the gene encoding the toxic protein is operatively linked to a Lac operator, such as is shown in the examples that follow. In other embodiments, other systems can be used to provide temperature sensitive regulation, such as the temperature-sensitive λ repressor CI857, or lactic acid bacteriophage temperature-inducible gene expression systems. Such regulatory elements and their sequences are known to those of skill in the art. Any temperature-sensitive repressor/operator system, or temperature-sensitive protein could be used instead of the temperature-sensitive lac operator system described.

Any suitable donor nucleic acid, including, but not limited to plasmid vectors, PCR products, or synthetic nucleic acids, that can be introduced into cells and which contain homologous recombination arms, may be used as the donor nucleic acid. The donor nucleic acid vector comprises a donor gene, which may be any gene of interest for inserting into the recipient viral vector, including but not limited to genes encoding therapeutic proteins and/or antigens for vaccine production such as Alpha-1 antitrypsin, Angiostatin, Antihemolytic factor, antibodies, Apolipoprotein, Apoprotein, Atrial natriuretic factor, Atrial natriuretic polypeptide, Atrial peptides, C—X—C chemokines (e.g., T39765, NAP-2, ENA-78, Gro-a, Gro-b, Gro-c, GCP-2, NAP-4, SDF-1, PF4, MIG), Calcitonin, CC chemokines (e.g., Monocyte chemoattractant protein-1, Monocyte chemoattractant protein-2, Monocyte chemoattractant protein-3, Monocyte inflammatory protein-1 alpha, Monocyte inflammatory protein-1 beta, RANTES, I309, R83915, R91733, HCC1, T58847, D31065, T64262), CD40 ligand, C-kit Ligand, Collagen, Colony stimulating factor (CSF), Complement factor 5a, Complement inhibitor, Complement receptor 1, cytokines, (e.g., epithelial Neutrophil Activating Peptide-78, GRO.alpha./MGSA, GROf-.beta., GRO.gamma., MIP-1.alpha., MIP-1.delta., MCP-1), Epidermal Growth Factor (EGF), Erythropoietin (“EPO”), Exfoliating toxins A and B, Factor IX, Factor VII, Factor VIII, Factor X, Fibroblast Growth Factor (FGF), Fibrinogen, Fibronectin, G-CSF, GM-CSF, Glucocerebrosidase, Gonadotropin, growth factors, Hedgehog proteins (e.g., Sonic, Indian, Desert), Hemoglobin, Hepatocyte Growth Factor (HGF), Hirudin, Human serum albumin, Insulin, Insulin-like Growth Factor (IGF), interferons (e.g., IFN-.alpha., IFN-.beta., IFN-.gamma.), interleukins (e.g., IL-1, IL-2, IL-3, IL-4, IL-5, IL-6, IL-7, IL-8, IL-9, M-10, IL-11, IL-12, etc.), Keratinocyte Growth Factor (KGF), Lactoferrin, leukemia inhibitory factor, Luciferase, Neurturin, Neutrophil inhibitory factor (NIF), oncostatin M, Osteogenic protein, Parathyroid hormone, PD-ECSF, PDGF, peptide hormones (e.g., Human Growth Hormone), Pleiotropin, Protein A, Protein G, Pyrogenic exotoxins A, B, and C, Relaxin, Renin, SCF, Soluble complement receptor I, Soluble I-CAM 1, Soluble interleukin receptors (IL-1, 2, 3, 4, 5, 6, 7, 9, 10, 11, 12, 13, 14, 15), Soluble TNF receptor, Somatomedin, Somatostatin, Somatotropin, Streptokinase, Superantigens, i.e., Staphylococcal enterotoxins (SEA, SEB, SECT, SEC2, SEC3, SED, SEE), Superoxide dismutase (SOD), Toxic shock syndrome toxin (TSST-1), Thymosin alpha 1, Tissue plasminogen activator, Tumor necrosis factor beta (TNF beta), Tumor necrosis factor receptor (TNFR), Tumor necrosis factor-alpha (TNF alpha), Vascular Endothelial Growth Factor (VEGEF), Urokinase and many others. As will be understood by those of skill in the art, the donor gene may comprise some/all regulatory elements necessary to promote expression of the donor gene after recombination with the recipient viral vector; alternatively, some/all of the regulatory sequences to promote expression of the donor gene may be provided by the recipient viral vector after recombination.

The recipient viral vector comprises two recombination sites flanking the gene encoding the toxic protein and the temperature sensitive regulatory system, wherein the two recombination sites are unique in the recipient viral vector, while the donor nucleic acid vector comprises two recombination sites flanking the donor gene, wherein the two recombination sites are unique in the donor nucleic acid vector, and are identical to the two recombination sites in the recipient viral vector. Recombination sites can occur anywhere within the intergenic regions that flank the recipient locus in the recipient viral vector genome. The intergenic regions are the physiological sequences present in the genome of the parental virus. The donor nucleic acid vector will be constructed to contain the same flanking region sequences.

The recipient virus contains a DNA polymerase that can induce recombination between sites on the virus genome and homologous DNA or homologous sites on a plasmid PCR product or synthetic DNA. Any suitable DNA polymerase can be used, including but not limited to Pox virus DNA polymerase, including but not limited to a vaccinia virus DNA polymerase. In one embodiment, the DNA polymerase is expressed from a gene integrated into the host cell chromosome or episome. In a preferred embodiment, the DNA polymerase is encoded by a gene present on the recipient viral vector.

Any suitable first and second temperatures can be used that are compatible with a given toxic protein. In a preferred embodiment, the first temperature is about 31° C. and the second temperature is between about 37° C. to about 39° C.

The recipient viral vector and donor nucleic acid vector may comprise any further functional units appropriate for a given purpose. In one embodiment of any of the above embodiments, the recipient viral vector may further comprise a gene encoding a detectable marker, to facilitate detection of recombination events.

In a third aspect, the present invention provides nucleic acid cassettes, comprising:

(a) a gene encoding a toxic protein;

(b) a regulatory element operatively linked to the gene encoding the toxic protein; and

(c) a temperature sensitive repressor of the regulatory element.

These cassettes can be used, for example, in creating the vectors for use in the methods of the present invention. In a preferred embodiment, the regulatory element comprises a lac operator and a temperature sensitive lac repressor. In a further preferred embodiment, the toxic protein is the PKR protein. In a further embodiment, at least the gene encoding the toxic gene and the temperature sensitive regulatory system are located between recombination sites that are unique in the cassette. The cassette may further comprise a gene encoding a marker, including but not limited to lacZ. Further embodiments of the cassettes of the invention are provided herein. The invention further provides recombinant vectors comprising the cassettes of the invention.

In a fourth aspect, the present invention provides recombinant viral vectors, comprising

(a) a gene encoding a toxic protein, wherein expression of the toxic protein is temperature sensitive; and

(b) two recombination sites flanking the gene encoding the toxic protein, wherein the two recombination sites are unique in the recipient viral vector.

In further embodiments, the recombinant viral vectors comprise:

(c) a regulatory element operatively linked to the gene encoding the toxic protein; and

(d) a temperature sensitive repressor of the temperature sensitive regulatory system.

In a preferred embodiment, the temperature sensitive regulatory element comprises a lac operator and the temperature sensitive repressor is the lac operator. In a further preferred embodiment, the toxic protein is the PKR protein. In a further embodiment, at least the gene encoding the toxic gene and the temperature sensitive regulatory element are located between recombination sites that are unique in the viral vector. The viral vector may further comprise a gene encoding a marker, including but not limited to lacZ. Further embodiments of the viral vectors of the invention are provided herein.

In a further embodiment, the vector comprises or consists of the nucleic acid of SEQ ID NO:1.

In a fifth aspect, the present invention provides recombinant viral particles comprising the recombinant viral vectors of the invention. In a further aspect, the present invention provides host cells comprising the recombinant virus of the invention. Further embodiments of the viral particles and host cells of the invention are provided herein.

### Examples

Our current methods of selection rely on requiring the parental virus genome to incorporate a new gene that will protect the host cells from toxicity of reagents we then add to the cells. The concept has always been to eliminate the host cells in which the parental virus replicates. Conversely, recombinant viruses which have taken up a resistance gene rescue the host cells from toxicity and are thus able to replicate. Since these toxic reagents can be harmful to humans, the current selection methods are not acceptable for use in constructing human vaccines.

The method of the present invention is a shift in concept: the parental virus itself is nonviable unless it has undergone recombination to replace the DNA encoding a toxic system, with DNA encoding a target antigen. There are no substances that are toxic to the cells, and no substances that increase the rate of mutation in the virus. There are no materials utilized that pose a risk to humans; the entire selection is for replication and can be carried out at 37° C. Yet the time required to acquire the recombinant virus that expresses the target antigen is a fraction of that needed for the methods currently used that are non-selective screening.

The methodology is based on using a virus with a conditionally toxic gene for negative selection and temperature regulation of the toxic gene. In an exemplary embodiment, the gene is not transcribed at 31° C. due to regulation by a temperature sensitive (ts) lac repressor, while the toxic gene is expressed at 37-39° C., because at that temperature the ts lac repressor is inactive. The virus, therefore, cannot survive at restrictive temperature unless it undergoes recombination with a plasmid that contains a gene encoding an antigen of interest; recombination will result in insertion of the target gene in place of the toxic gene. Thus, parental virus can be amplified at 31° C., but are selected against at 37° C. For example, a cassette can be is inserted into the thymidine kinase (TK) locus of the virus; the virus with this cassette is viable at 31° C., but nonviable at 37-39° C. The virus is rescued at the restrictive temperature if in vivo recombination takes place, exchanging the cassette containing a gene toxic to the virus for a gene that encodes the target antigen.

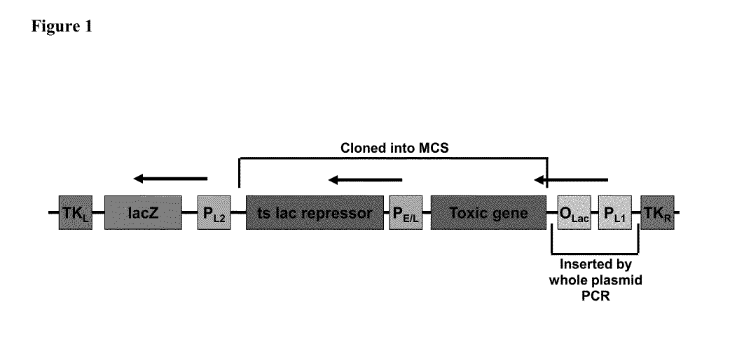

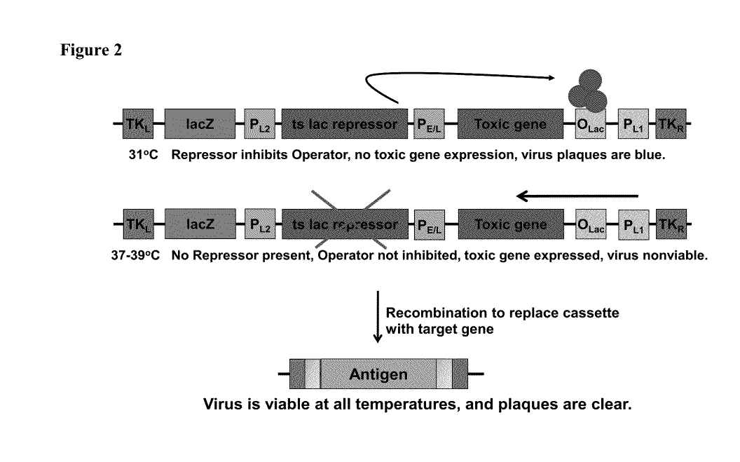

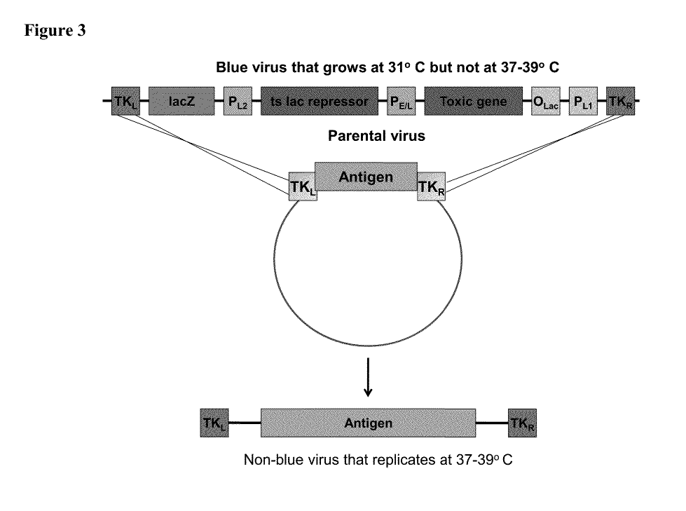

The starting material is a transfer plasmid containing the screening marker lacZ positioned between the left and right flanking arms of the TK locus. By whole plasmid PCR, and standard cloning, each section of the cassette is inserted into the transfer plasmid (FIG. 1). The cassette contains 1) a gene toxic to the virus, that is regulated by 2) a lac operator, 3) a ts lac repressor, and 4) a marker gene (lacZ). Expression of the toxic gene is regulated by the lac operator. At 31° C. therefore, the binding of the ts lac repressor to the operator inhibits expression of the toxic gene while the virus is being cultivated in cells at 31°. The lac repressor is temperature sensitive, so that at 37-39 C it is not functional. In the absence of the repressor, expression of the toxic gene occurs, which then makes the virus nonviable at 37-39° (See FIG. 2). If the virus successfully recombines with a plasmid carrying the gene of interest, the toxic cassette is deleted, and the gene of interest takes its place; the virus is now viable at 37-39 C. In vivo recombination takes place between homologous regions of DNA, such as the genomic flanking arms of the TK locus and homologous TK flanking arms on either side of the gene of interest in the transfer plasmid (See FIG. 3). Loss of lacZ (non-blue virus) also screens against a mutation in the toxic gene of the cassette which could result in viability at 37° C. even in the absence of recombination.

Through previous studies of vaccinia virus, we know that overexpression of PKR is lethal to vaccinia virus. Therefore, only those viruses that have successfully recombined with the plasmid, and replaced the toxic cassette with the DNA encoding the target gene, will survive. Alternatively, the temperature sensitive toxic cassette can encode a protein that is functional (toxic) at one temperature but not at another.

An exemplary recombinant viral vector according to the present is presented in SEQ ID NO:1. This construct is referred to as temperature sensitive construct cm5 and includes nucleotides encoding a TK flanking regions, a temperature sensitive lac repressor, a PKR toxic protein, lacZ, and various regulatory elements.

We claim:

1. A method for insertion of genetic material into genomic DNA comprising
(a) incubating host cells comprising recipient viral vector and donor nucleic acid vector under conditions and for a time suitable for promoting recombination between the recipient viral vector and the donor nucleic acid vector, wherein
(i) the recipient viral vector comprises
(A) a gene encoding a toxic protein, wherein expression of the toxic protein is temperature sensitive; or the toxic protein itself is only toxic at a particular temperature; and
(B) two recombination sites flanking the gene encoding the toxic protein, wherein the two recombination sites are unique in the recipient viral vector; wherein
(ii) the donor nucleic acid vector comprises
(A) a donor gene; and
(B) two recombination sites flanking the donor gene, wherein the two recombination sites are (I) unique in the donor nucleic acid vector, and (II) identical to the two recombination sites in the recipient viral vector; and wherein
(iii) the host cell comprises a promiscuous DNA polymerase capable of synthesizing recipient viral vector DNA, and
wherein the conditions comprise incubating the host cells at a first temperature at which the toxic protein is not expressed;
(b) subsequently culturing the host cells at a second temperature at which the toxic protein is expressed by the recipient viral vector; and
(c) selecting viruses that grow at the second temperature, wherein viruses that grow at the second temperature are recombinant recipient viral vectors in which the donor gene has been inserted into the recipient viral vector, replacing the toxic gene.

2. The method of claim 1, wherein the host cell is a eukaryotic cell.

3. The method of claim 1, wherein the host cell is a mammalian cell.

4. The method of claim 1, wherein the recipient viral vector comprises a Pox viral genome.

5. The method of claim 1, wherein the recipient viral vector comprises a vaccinia viral genome.

6. The method of claim 1, wherein the toxic protein is selected from the group consisting of protein kinase R (PKR), DNAses, RNAses, restriction endonucleases, proteases, and proteins that induce apoptosis.

7. The method of claim 1, wherein expression of the gene encoding the toxic protein is controlled by a heterologous temperature sensitive regulatory system, wherein the temperature sensitive element is present, or is encoded by a nucleic acid present, between the two recombination sites.

8. The method of claim 7, wherein the heterologous temperature sensitive regulatory system comprises a system selected from the group consisting of (i) temperature sensitive lac repressor, wherein the gene encoding the toxic protein is operatively linked to a Lac operator, (ii) temperature-sensitive λ repressor CI857, and (iii) lactic acid bacteriophage temperature-inducible gene expression systems.

9. The method of claim 1 wherein the donor nucleic acid vector comprises a donor gene encoding a therapeutic protein and/or an antigen for vaccine production.

10. The method of claim 1, wherein the DNA polymerase is a Pox virus DNA polymerase.

11. The method of claim 1, wherein the DNA polymerase is a vaccinia virus DNA polymerase.
